(19) United States
(12) Patent Application Publication
(10) Pub. No.: US 2008/0275459 A1
(43) Pub. Date: Nov. 6, 2008

(54) Surgical instrument attachment mechanism

(76) Inventors: Charles Anthony Dickinson, Millington, TN (US); William A. Rezach, Atoka, TN (US); Daniel Roberts, Germantown, TN (US)

Correspondence Address: KRIEG DEVAULT LLP, ONE INDIANA SQUARE, SUITE 2800, INDIANAPOLIS, IN, 46204-2709, US

(21) Appl. No.: 11/799,662

(22) Filed: May 2, 2007

(51) Int. Cl. A61B 17/88

(52) U.S. Cl. 606/104

(57) ABSTRACT

An instrument attachment mechanism is disclosed that is operable to be temporarily, but securely, connected with a surgical device. The attachment mechanism includes a body that has a drive member protruding outwardly from a portion of the body. The drive member includes a plurality of side walls and a passageway running from a bottom surface of the drive member to a predetermined depth in the drive member. At least one slot is located in a respective one of the plurality of side walls that extends through the side wall into the passageway. A biasing member is positioned within the passageway such that a select portion of the biasing member protrudes outwardly from the slot. A surgical device, such as a surgical instrument or implant, may be temporarily connected with the drive member such that the select portions of the biasing member apply force to an inner wall of a drive receiving member of the surgical device.

### TECHNICAL FIELD

The present invention relates generally to the field of surgical instrumentation and methods, and more particularly relates to instrumentation and methods for temporarily interconnecting surgical devices to other surgical devices while at the same time maintaining a secure connection with the respective device.

### BACKGROUND

During surgical procedures it is not uncommon to use a plurality of instruments to complete the surgical procedure. For example, during spinal surgery a plurality of bone screws may be inserted into a patient's spine. The bone screw may include a drive receiving member that is used by a tool to secure the bone screw into the bone tissue. Once in position in the bone tissue, the tool may be removed so that other instruments may be attached to the drive receiving member. Other surgical devices may then be temporarily connected with the bone screw so that other surgical steps may be performed.

Currently, these surgical devices are connected by inserting a drive member of the surgical device into the drive receiving member of the bone screw. The drive receiving member may comprise a hexagonal aperture, for example, located on an upper surface of the bone screw that acts as a female engagement member. The drive member may comprise a male hexagonal shaped member that is designed to be inserted into the female hexagonal shaped member. The problem with this arrangement is that the male hexagonal shaped member may inadvertently slip out of or be removed from the female hexagonal shaped member. As such, a need exists for an apparatus that will temporarily interconnect a female drive receiving member with a male drive member while at the same time maintaining a secure connection with the female drive receiving member.

### SUMMARY

One aspect of the present invention discloses an apparatus that may be used to temporarily interconnect two respective surgical devices to one another while maintaining a firm or secure grip with at least one of the respective surgical devices. The apparatus includes a body having at least one drive member that protrudes outwardly from a portion of the body. The drive member includes a plurality of side walls and an inner passageway or aperture that extends from a bottom surface of the drive member to a predetermined depth within an inside diameter of the drive member. At least one oval shaped slot is located in a respective side wall that extends inwardly to the passageway. A superelastic biasing member is located or positioned within the passageway such that a select portion of the superelastic biasing member protrudes outwardly from the slot above an outer surface of the side wall.

A surgical device that includes a drive receiving member is connected with the drive member. When connected, the superelastic biasing member moves to a compressed state such that a predetermined amount of force is applied to an inner wall of the drive receiving member of the surgical device. The predetermined amount of force forms a friction fit between the surgical device and the drive member thereby securely holding the drive member and the surgical device together. Upon application of a predetermined amount of force to the body in a direction away from the surgical device, the drive member will detach from the drive receiving member. The amount of force necessary to disconnect the two respective devices is an amount required to overcome the friction fit formed by the biasing member, which will vary depending upon the makeup of the biasing member.

The body of the apparatus may also include a second drive member having a biasing member positioned therein on a distal end of the body. In alternative embodiments, the distal end of the body may include an interconnect member. The interconnect member may include a drive receiving aperture that extends within the body a predetermined distance. The drive receiving aperture allows a second surgical device to be detachably connected with the body. The drive member may be shaped in one of several different shapes commonly used for drive members. The drive receiving aperture may also be shaped in one of several different shapes that are designed to mate with the drive members in a male/female engagement fashion.

Another aspect of the present invention discloses a method of temporarily interconnecting surgical devices. The method includes the step of providing an attachment mechanism including a drive member having a passageway in an inner portion of the drive member. The drive member also includes a pair of opposing slots in side walls of the drive member that extend to the passageway. A biasing member is located or positioned in the passageway such that select portions of the biasing member protrude outwardly from the pair of opposing slots. The method also includes the step of connecting the drive member with a drive receiving member of a surgical device such that the select portions of the biasing member that protrude outwardly from the pair of opposing side walls apply a select amount of force to a respective pair of inner walls of the surgical device. In an alternative embodiment, the biasing member may be inserted into the slot, thereby eliminating the need for passageway. In this embodiment, the slot runs through the drive member from one respective sidewall to an opposing sidewall.

The method may also include the step of providing a drive receiving aperture on a distal end of the attachment mechanism. The drive receiving member allows a second surgical device to be temporarily connected with the attachment mechanism. The surgical devices may comprise a bone screw, a screw extender, a surgical instrument, or any other apparatus commonly used during surgical procedures. The select portions of the biasing member positioned in the drive member form a friction fit between the inner walls of the drive receiving member of the surgical instrument. The biasing member is formed from a superelastic material that when compressed, as is the case when the drive member is inserted into the drive receiving member, tends to still want to maintain its original shape in an uncompressed state. As such, the select portions of the biasing member that protrude outwardly from the slots apply force to the inner walls of the drive receiving member thereby securely holding the drive member in the drive receiving member. If a predetermined amount of force is applied to the attachment mechanism in a direction away from the surgical device, the drive member may be disconnected from the surgical device.

Yet another aspect of the present invention discloses a tool for detachably engaging a surgical device. The tool includes a handle portion that is connected with a shaft portion. The shaft portion is also connected with an attachment mechanism. The attachment mechanism includes a body having a drive member protruding outwardly from a portion of the body. The drive member includes a plurality of side walls and an inner passageway that extends from a bottom surface of the drive member to a predetermined depth in the drive member. At least one slot is located in a respective side wall that extends inwardly to the passageway. A biasing member, which is formed of a superelastic material, is positioned within the passageway such that a select portion of the biasing member protrudes outwardly from the slot above an outer surface of the respective side wall.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is a perspective view of a representative attachment mechanism;

FIG. 2 is a side view of the representative attachment mechanism depicted in FIG. 1;

FIG. 3 is a cross-sectional view of the attachment mechanism along section 3-3 set forth in FIG. 2;

FIG. 4 is a side view of the attachment mechanism interconnecting two respective medical devices or instruments;

FIG. 5 is a cross-sectional view of the assembly illustrated in FIG. 4 along section 5-5 set forth in FIG. 4;

FIG. 6 is a cross-sectional side view of a portion of the assembly illustrated in FIG. 5;

FIG. 7 is a cross-sectional side view of another representative attachment mechanism;

FIG. 8 is a cross-sectional side view of a tool including a representative attachment mechanism; and

FIG. 9 is a cross-sectional side view of yet another representative attachment mechanism.

### DETAILED DESCRIPTION

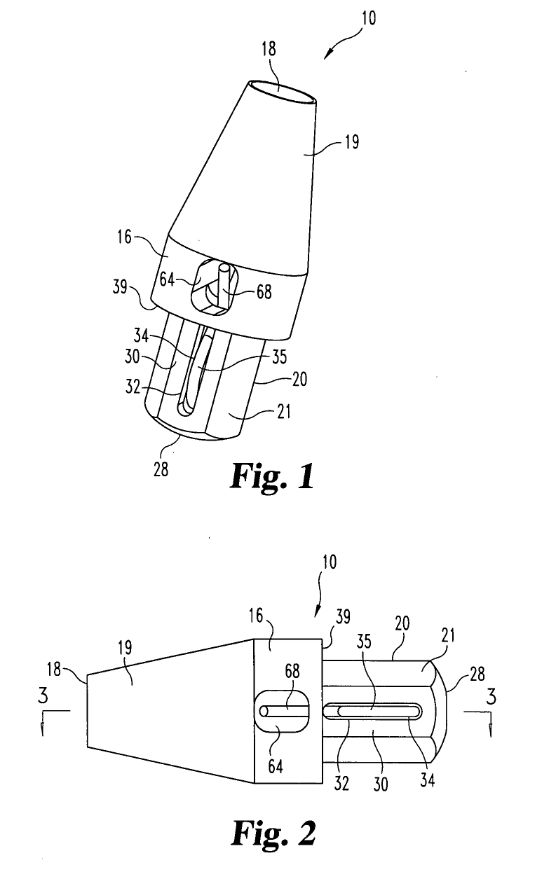

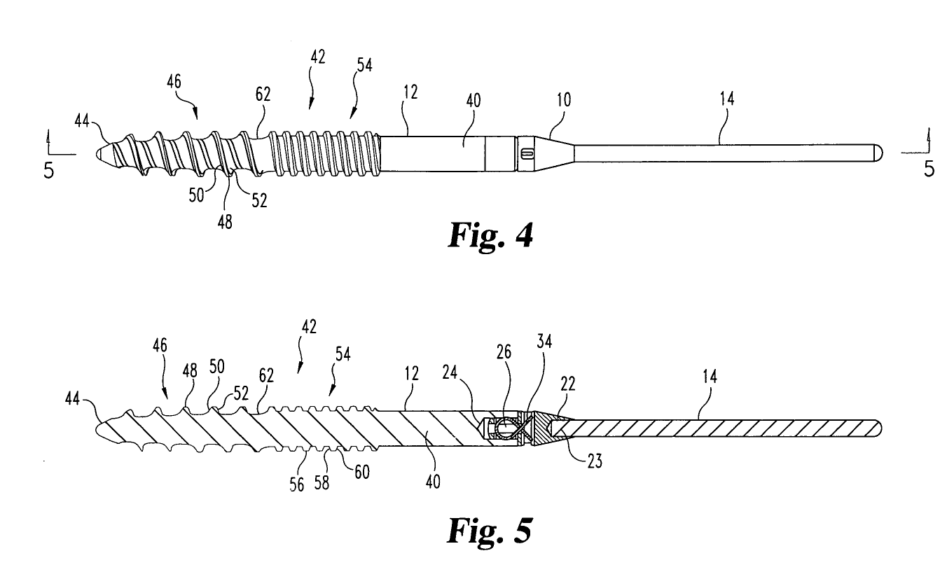

Referring collectively to FIGS. 1-4, an instrument attachment mechanism 10 is disclosed that is used to temporarily interconnect a first surgical device 12 with a second surgical device 14. In the embodiment illustrated in FIG. 4, the first surgical device 12 is illustrated as a bone screw. Although a bone screw is illustrated in the representative embodiment, it should be appreciated that the present invention may be utilized with various types of surgical devices. For example, the surgical device may comprise a surgical implant, a surgical tool, a surgical instrument, or any other type of surgical device designed to be connected with other devices. Further, the second surgical device 14 is illustrated as a screw extender, but as with the bone screw, it should be appreciated that the present invention may be utilized in conjunction with various other surgical elements, instruments, and tools. As such, the illustration of a bone screw 12 and a screw extender 14 throughout the various figures should be viewed in an illustrative sense, rather than in a restrictive sense, unless otherwise claimed.

Referring to FIGS. 1-3, the attachment mechanism 10 includes a body 16 that has an interconnect member 18 located at a distal end 19 of the body 16 and a second interconnect member 20 located at a proximate end 21 of the body 16. The second surgical device 14 is detachably connected with the interconnect member 18 and the first surgical device 12 is detachably connected with the drive member 20. During a surgical procedure, the attachment mechanism 10 is detachably connected with the first surgical device 12 so that the second surgical device 14 may then be detachably connected with the first surgical device 12 via the drive member 20.

The interconnect member 18 of the attachment mechanism 10 includes an aperture 22 that removably receives a proximate end or connection portion 23 of the second surgical device 14, which is best illustrated in FIG. 5. The aperture 22 extends into or within the body 16 of the attachment mechanism 10 a predetermined depth from the distal end 19 of the body 16. The aperture 22 extends downwardly from a top surface of the body 16. Although the aperture 22 is illustrated as a circular-shaped aperture, it should be appreciated that other shaped apertures, such as, for example, a hex, slotted, square, triangular, pozidriv, Torx, Phillips, Robertson, tri-wing, torq-set, and spanner head shaped aperture, may also be utilized in alternative embodiments. The proximate end 23 of the second surgical device 14 is shaped to be removably inserted into the aperture 22. As such, the interconnect member 18 and the proximate end 23 of the second surgical device 14 are designed to be removably engaged with one another in a male/female engagement manner. In alternative embodiments, the male member may be located on the body 16 of the attachment mechanism 10 and the female member may be located on the second surgical device 14.

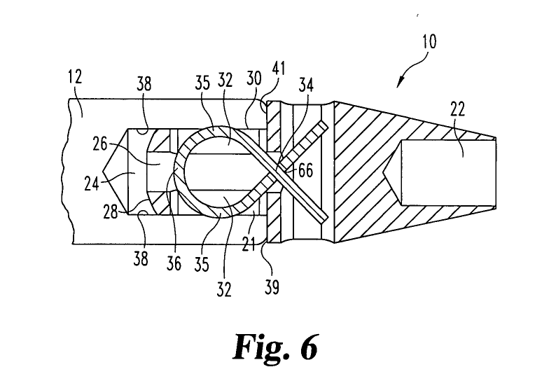

Referring collectively to FIGS. 1-6, the drive member 20 protrudes downwardly from the body 16 a predetermined distance and is designed to be removably inserted into a drive receiving aperture 24 located on a proximate end of the first surgical device 12. The drive member 20 includes a passageway 26 positioned within a portion of the drive member 20. The passageway 26 begins at a bottom surface 28 of the drive member 20 and runs longitudinally through an inside diameter of the drive member 20 a predetermined distance into the body 16 of the attachment mechanism 10. At least one, but preferentially two, side surfaces 30 of the drive member 20 include a slot 32 that runs through the drive member 20 into the passageway 26. Although the drive member 20 is illustrated as having a hex shape, it should be appreciated that various other shaped interconnect members, such as, for example, a square, triangular, pozidriv, Torx, Phillips, Robertson, tri-wing, torq-set, and spanner-head shaped member, may be utilized in alternative embodiments of the present invention.

The attachment mechanism 10 includes a biasing member 34 that is used to secure the drive member 20 within the drive receiving aperture 24 of the first surgical device 12. The biasing member 34 is positioned in the passageway 26 of the drive member 20 and, in one embodiment, comprises a superelastic wire. The biasing member 34 is positioned in the passageway 26 of the drive member 20 such that select portions 35 of the biasing member 34 protrude outwardly through the slots 32 in the sidewalls 30 of the drive member 20. The select portions 35 of the biasing member 34 extend outwardly a predetermined distance from the sidewalls 30 of the drive member 20 such that when the drive member 20 is inserted into the drive receiving aperture 24 the select portions make contact with inner walls 38 of the first surgical device 12.

During assembly of the attachment mechanism 10, the biasing member 34 is inserted into the passageway 26 by placing the biasing member 34 in a compressed state. Once in the compressed state, the biasing member 34 may be slid into the passageway 26 of the drive member 20. The biasing member 34 is slid into the passageway 26 by accessing the passageway 26 at the bottom surface 28 of the attachment mechanism 10. Once the biasing member 20 has been slid into the passageway 26 to a predetermined depth, the select portions 35 of the biasing member 34 springs or snaps into place such that the select portions 35 of the biasing member 34 protrude outwardly through the slots 32 in the drive member 20. In an alternative embodiment, the biasing member 34 may be compressed and inserted into the slot 32, thereby eliminating the need for passageway 26. In this embodiment, the slot 32 runs through one sidewall 30 to an opposing sidewall 30.

In one embodiment of the present invention, the biasing member 34 is at least partially formed of a shape-memory material that exhibits pseudoelastic characteristics or behavior at about human body temperature, the details of which will be discussed below. It should be understood that the terms “pseudoelastic” and “superelastic” have identical meanings and are used interchangeably throughout this document. In one embodiment of the present invention, the entire biasing member 34 is formed of the shape-memory material. However, it should be understood that the biasing member 34 may also be formed using any suitable biocompatible material, such as, for example, stainless steel or titanium.

A shape-memory alloy (“SMA”) is an alloy that exhibits a “shape-memory” characteristic or behavior in which a particular component formed of a shape-memory alloy is capable of being deformed from an initial “memorized” shape or configuration to a different shape or configuration, and then reformed back toward its initial shape or configuration. The ability to possess shape-memory is a result of the fact that the SMA undergoes a reversible transformation from an austenitic state to a martensitic state. If this transformation occurs due to a change in temperature, the shape-memory phenomena is commonly referred to as thermoelastic martensitic transformation. However, if the martensitic transformation occurs due to the imposition of stress or force, the shape-memory phenomena is commonly referred to as stress-induced martensitic transformation. The present invention is primarily concerned with stress-induced martensitic transformation.

SMAs are known to display a superelastic phenomena or rubber-like behavior in which a strain attained beyond the elastic limit of the SMA material during loading is recovered during unloading. This superelastic phenomenon occurs when stress is applied to an SMA article at a temperature slightly higher than the temperature at which the SMA begins to transform into austenite (sometimes referred to as the transformation temperature). When stressed, the article first deforms elastically up to the yield point of the SMA material (sometimes referred to as the critical stress). However, upon the further imposition of stress, the SMA material begins to transform into stress-induced martensite or “SIM”. This transformation takes place at essentially constant stress, up to the point where the SMA material is completely transformed into martensite. When the stress is removed, the SMA material will revert back into austenite and the article will return to its original, pre-programmed or memorized shape. This phenomenon is sometimes referred to as superelasticity or pseudoelasticity. It should be understood that this phenomena can occur without a corresponding change in temperature of the SMA material. Further details regarding the superelastic phenomena and additional characteristics of SIM are more fully described by Yuichi Suzuki in an article entitled Shape Memory Effect and Super-Elasticity in Ni—Ti Alloys, Titanium and Zirconium, Vol. 30, No. 4, October 1982, the contents of which are hereby incorporated by reference.

There is a wide variety of shape-memory materials suitable for use with the present invention, including shape-memory metal alloys (e.g., alloys of known metals, such as, for example, copper and zinc, nickel and titanium, and silver and cadmium) and shape-memory polymers. While there are many alloys which exhibit shape-memory characteristics, one of the more common SMAs is an alloy of nickel and titanium. One such alloy is nitinol, which is a bio-compatible SMA formed of nickel and titanium. Nitinol is well suited for the particular application of the present invention because it can be programmed to undergo a stress-induced martensitic transformation at about normal human body temperature (i.e., at about 35-40 degrees Celsius). Moreover, nitinol has a very low corrosion rate and excellent wear resistance, thereby providing an advantage when used as a support structure within the human body. Additionally, implant studies in animals have shown minimal elevations of nickel in the tissues in contact with the nitinol material. It should be understood, however, that other SMA materials that exhibit superelastic characteristics are contemplated as being within the scope of the invention.

Referring collectively to FIGS. 4-6, the drive member 20 detachably interconnects the first surgical device 12 with the attachment mechanism 10. When the drive member 20 is inserted into the drive receiving aperture 24 of the first surgical device 12, the select portions 35 of the biasing member 34 that protrude outwardly through the slots 32 apply a predetermined amount of force to inner walls 38 of the drive receiving aperture 24. The application of force to the inner walls 38 of the drive receiving aperture 24 forcibly maintains the drive member 20 within the drive receiving aperture 24.

As set forth above, since the biasing member 34 is made from a superelastic SMA in some embodiments, the biasing member 34 naturally wants to return to its uncompressed state, which when compressed by application of stress to the select portions 35 of the biasing member 34, causes the select portions 35 of the biasing member 34 that protrude outwardly through the slots 32 to want to push outwardly thereby applying force to the inner walls 38 of the receiving aperture 24. The select portions 35 of the biasing member 34 form a friction fit between the drive member 20 of the attachment mechanism 10 and the first surgical device 12. The body 16 of the attachment mechanism 10 includes an abutment surface 39 that engages an upper surface 41 of the first surgical device 12 such that the drive member 20 only travels to a predetermined depth in the drive receiving aperture 24.

As previously set forth, the attachment mechanism 10 is designed to be detachably connected with the first surgical device 12. In order to remove the attachment mechanism 10 from the first surgical device 12, a predetermined amount of force is applied to the attachment mechanism 10 in a direction opposite or away from the first surgical device 12. The amount of force needed to detach the attachment mechanism 10 from the first surgical device 12 will varies depending upon the material, size, and shape of the biasing member 34. Once the attachment mechanism 10 is disconnected from the first surgical device 12, the select portions 35 of the biasing member 34 return to their uncompressed state.

Referring to FIGS. 4 and 5, in one embodiment of the present invention, the first surgical device 12 comprises a post screw. The post screw 12 may be formed of a titanium-aluminum alloy (Ti-6Al-4V) and include a proximal shaft portion 40 having a smooth outer surface and a threaded shank portion 42 extending distally from the proximal shaft portion 40. The post screw 12 is provided in a number of sizes and configurations, including varying lengths, diameters and thread arrangements. As previously set forth, the drive receiving aperture 24 may comprise a hexagonally-shaped tool engaging recess that is formed in the end of the proximal shaft portion 40 for receiving the drive member 20 of the attachment mechanism 10. The threaded shank portion 42 is provided with a tapered distal tip 44 to facilitate introduction into bone tissue.

The threaded shank portion 42 further includes a first lead thread portion 46 which is provided with a flat thread crest 48 and angled leading and trailing thread faces 50, 52. The threaded shank portion 42 also includes a second lead thread portion 54 which is provided with a arcuate thread crest 56 and second angled leading and trailing thread faces 58, 60. The first and second lead thread portions 46, 54 define a constant and uniform thread pitch. In some embodiments, the threaded shank portion 42 defines a uniform major thread diameter which runs out onto the smooth, proximal shaft portion 40. Additionally, in some embodiments of the post screw 12, a self-tapping feature 62 may be included that forms threads in bone tissue for the second lead thread portion 54.

As previously set forth, although the first surgical device 12 is illustrated as a double lead post screw, it should be appreciated that other surgical devices may benefit from the present invention. The attachment mechanism 10 may be utilized in conjunction with any surgical device, implant or apparatus that includes a drive receiving aperture 24 that is designed to receive a drive member. As such, the first surgical device 12, unless specifically claimed otherwise, should be broadly construed to include various types of devices, implants or apparatuses.

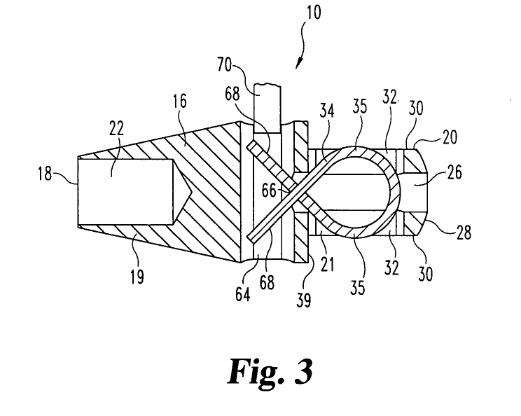

Referring back to FIGS. 1-3, in one representative embodiment the body 16 of the attachment mechanism 10 may include a second passageway 64 that runs horizontally through the width of the body 16. A portion of the second passageway 64 intersects with a portion of the first passageway 26 to form an opening 66. In this embodiment, the biasing member 34 is formed having a ribbon shape. When the ribbon shaped biasing member 34 is placed within the first passageway 26, upper portions 68 of the ribbon shaped biasing member 34 protrude through the opening 66 into the second passageway 64. Insertion of a tool 70, which is only partially illustrated in FIG. 3, into the second passageway 64 and application of force to both of the upper portions 68 of the ribbon shaped biasing member 34 in an inward direction cause the select portions 35 of the ribbon shaped biasing member 34 to retract into a compressed state, thereby allowing the attachment mechanism 10 to more readily be removed from the surgical implant member 12.

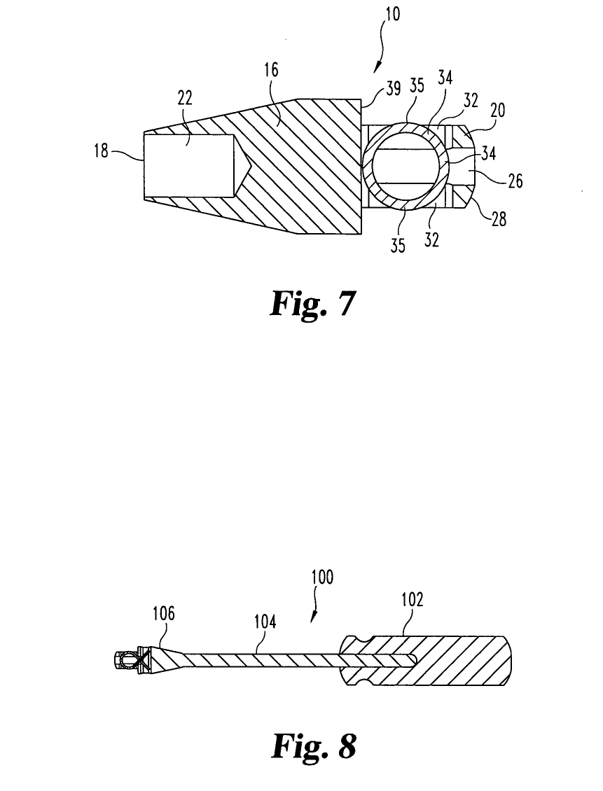

Referring to FIG. 7, in another embodiment of the present invention, the biasing member 34 of the attachment mechanism 10 may comprise a circular shaped biasing member. The circular shaped biasing member 34 is inserted into the first passageway 26 in a manner as previously set forth with respect to the embodiment set forth in FIGS. 1-3. In an alternative embodiment, the biasing member 34 may be inserted into the slot 32, thereby eliminating the need for passageway 26. Other than having a circular shape as opposed to a ribbon shape, the attachment mechanism 10 utilizing the circular shaped biasing member 34 performs the same function as previously set forth with respect to the embodiment set forth in FIGS. 1-3. As such, a detailed discussion of the operation of this respective attachment mechanism 10 is not set forth herein for the sake of brevity.

Referring to FIG. 8, yet another embodiment of the present invention discloses a temporary attachment tool 100. The temporary attachment tool 100 includes a handle portion 102, a shaft portion 104, and an attachment mechanism portion 106. The handle portion 102 is connected with, or may be formed as an integral part of, the shaft portion 104. The shaft portion 104 is connected with, or may be formed as an integral part of, the shaft portion 104 as well. The attachment mechanism portion 106 of the temporary attachment tool 100 is designed the same as the attachment mechanisms 10 set forth in detail above. As such, a detailed discussion of the components and functionality of the attachment mechanism portion 106 of the temporary attachment tool 100 have been omitted for the sake of brevity as well.

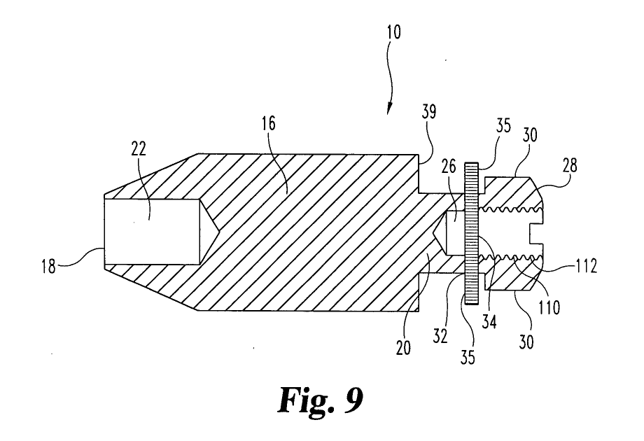

Referring to FIG. 9, another representative embodiment of an attachment mechanism 10 is disclosed that includes a body 16 and a drive member 20. The drive member 20, like in the previous embodiments, includes a first passageway 26 located on a bottom surface 28 of the drive member 20 and runs a predetermined depth into the drive member 20. A pair of opposing slots 32 is located in the side walls 30 of the drive member 20. A biasing member 34 is positioned in the passageway 26 such that select portions 35 of the biasing member 34 protrude outwardly through the slots 32 beyond the side walls 30. In this embodiment, an end portion 110 of the passageway 26 includes a threaded portion 112 that runs to the slots 32. A setscrew 114 is screwed into the threaded portion 112 to further secure the biasing member 34 within the passageway 26.

While the invention has been illustrated and described in detail in the drawings and foregoing description, the same is to be considered as illustrative and not restrictive in character.

What is claimed is:

1. A surgical apparatus, comprising:
a body;
a drive member protruding outwardly from a portion of said body, wherein said drive member includes a plurality of side walls and an inner passageway extending from a bottom surface of said drive member to a predetermined depth in said drive member;
at least one slot located in a respective side wall extending inwardly to said passageway; and
a biasing member positioned within said passageway such that a select portion of said biasing member protrudes outwardly from said at least one slot above an outer surface of said respective side wall.

2. The apparatus of claim 1, wherein said biasing member comprises a superelastic material.

3. The apparatus of claim 2, wherein said superelastic material comprises nitinol.

4. The apparatus of claim 1, further comprising a surgical device connected with said drive member, wherein said surgical device includes a drive receiving member for receiving said drive member.

5. The apparatus of claim 4, wherein said drive receiving member includes a plurality of inner walls that selectively engage respective side walls of said drive member such that said biasing member is placed in a compressed state thereby applying force to said inner wall of said drive receiving member to form a friction fit between said inner wall and at least one respective side wall.

6. The apparatus of claim 1, further comprising a threaded portion in said inner passageway running to said at least one slot.

7. The apparatus of claim 6, further comprising a set screw threadably engaged with said threaded portion to secure said biasing member in said passageway.

8. The apparatus of claim 1, further comprising an interconnect member located on a distal end of said body.

9. The apparatus of claim 8, further comprising a surgical device detachably connected with said interconnect member.

10. The apparatus of claim 1, wherein said biasing member is ribbon shaped.

11. The apparatus of claim 1, wherein said biasing member is circular shaped.

12. A method, comprising:
providing an attachment mechanism including a drive member having a passageway in an inner portion of said drive member and a pair of opposing slots in side walls of said drive member extending to said passageway, wherein a biasing member is located in said passageway such that select portions of said biasing member protrude outwardly from said pair of opposing slots; and
connecting said drive member with a drive receiving member of a surgical device such that said select portions of said biasing member that protrude outwardly from said pair of opposing side walls apply a select amount of force to a respective pair of inner walls of said surgical device.

13. The method of claim 12, further comprising applying a predetermined amount of force to said attachment mechanism in a direction away from said surgical device such that said select portions of said biasing member release said drive member from said drive receiving member.

14. The method of claim 12, wherein said surgical device comprises a bone screw.

15. The method of claim 12, further comprising providing an interconnect member located on a distal end of said attachment mechanism.

16. The method of claim 15, wherein said interconnect member includes a second drive receiving aperture.

17. The method of claim 16, further comprising inserting a second surgical device in said second drive receiving aperture of said interconnect member.

18. The method of claim 17, wherein said second surgical device comprises a screw extender.

19. The method of claim 12, wherein said surgical device comprises a surgical implant.

20. An apparatus, comprising:
a body having a drive member protruding downwardly from a portion of said body, said drive member including a plurality of side walls and an inner passageway extending from a bottom surface of said drive member to a predetermined depth in said drive member;
a pair of slots located in opposing side walls of said drive member extending inwardly to said passageway; and
a superelastic biasing member positioned within said passageway such that select portions of said superelastic biasing member protrude outwardly from said pair of slots above an outer surface of said opposing side walls.

21. The apparatus of claim 20, wherein said body includes an interconnect member positioned opposite said drive member including an aperture protruding downwardly from a top surface of said interconnect member.

22. The apparatus of claim 20, further comprising a surgical device connected with said drive member.

23. The apparatus of claim 22, wherein said surgical device includes a drive receiving member having a plurality of inner walls that selectively engage respective side walls of said drive member such that said superelastic biasing member is placed in a compressed state thereby causing said select portions to apply force to said inner walls of said drive receiving member.

24. The apparatus of claim 23, wherein said body includes an interconnect member positioned opposite said drive member including an aperture protruding downwardly from a top surface of said interconnect member.

25. The apparatus of claim 24, further comprising a second surgical instrument having a connection portion inserted into said aperture.

26. The apparatus of claim 20, further comprising a second passageway extending perpendicularly through said body in relation to said passageway and intersecting with a portion of said passageway to form an opening between the second passageway and the passageway.

27. The apparatus of claim 26, wherein said superelastic biasing member has a ribbon shape including respective upper portions that protrude through said opening into said second passageway.

28. The apparatus of claim 20, wherein said passageway includes a threaded portion extending to said pair of slots.

29. The apparatus of claim 28, wherein a set screw is positioned in said threaded portion to secure said superelastic biasing member within said passageway.

30. A surgical tool for detachably engaging a device, comprising:
a handle portion;
a shaft portion connected with said handle portion;
an attachment mechanism portion connected with said shaft portion;
wherein said attachment mechanism portion comprises:
a body;
a drive member protruding outwardly from a portion of said body, wherein said drive member includes a plurality of side walls and an inner passageway extending from a bottom surface of said drive member to a predetermined depth in said drive member;
at least one slot located in a respective side wall extending inwardly to said passageway; and
a biasing member positioned within said passageway such that a select portion of said biasing member protrudes outwardly from said slot above an outer surface of said respective side wall.

31. A surgical apparatus, comprising:
a body having a drive member protruding downwardly from a portion of said body, said drive member including a plurality of side walls;
a slot extending through said drive member from a first respective sidewall to an opposing sidewall; and
a biasing member positioned within said slot such that select portions of said biasing member protrude outwardly from said slot above an outer surface of said side walls.

32. The apparatus of claim 31, wherein said biasing member comprises a superelastic material.

33. The apparatus of claim 32, wherein said superelastic material comprises nitinol.

34. The apparatus of claim 31, wherein said biasing member is circular shaped.
